(19) United States
(12) Patent Application Publication
(10) Pub. No.: US 2022/0008601 A1
(43) Pub. Date: Jan. 13, 2022

(54) AIR PURIFIER

(71) Applicants: San-Hsi Wang, Taoyuan City (TW); Ching-Chung Wang, Taoyuan City (TW)

(72) Inventor: San-Hsi WANG, Taoyuan City (TW)

(21) Appl. No.: 16/927,783

(22) Filed: Jul. 13, 2020

(51) Int. Cl. A61L 9/20; B01D 46/00; B01D 46/24

(52) U.S. Cl. CPC A61L 9/20; B01D 46/0028; B01D 46/2411; A61L 2209/12; B01D 2279/40; A61L 2209/14; B01D 2279/65

(57) ABSTRACT

An air purifier includes a housing, a filter unit and a sterilization unit. The housing has an air outlet and an air inlet under the air outlet. A filtering chamber is formed in the housing by the air outlet and the air inlet. The filtering chamber has a first chamber and a second chamber. The filter unit is disposed in the first chamber. The sterilization unit is disposed in the filtering chamber. A fan is disposed in the second chamber of the housing and over the sterilization unit. The fan is used for sucking air outside the housing, passing the air through the filter unit and the sterilization unit and emitting the air. This can accomplish an object of air purification.

### BACKGROUND OF THE INVENTION

### Technical Field

The invention relates to filters, and particularly to air purifiers which filter air for human inhalation.

### Related Art

Because air pollution is becoming more and more serious, and infectious diseases between humans occasionally occur, infection via air or harmful materials in the air may affect the bodily health of many people or even make those who breathe it in ill. These hazards make people pay more attention to air quality.

Large air purifiers or air-conditioners with an air filtering function are usually used to improve air quality. However, such devices are only used indoors and are fixedly installed in buildings, so they are disadvantageous and inconvenient for personal use. For an individual person, wearing a mask is preferred, but its effect is limited.

In view of this, the inventors have devoted themselves to the above-mentioned prior art, researched intensively, and cooperated with the application of science to try to solve the above-mentioned problems. Thus, an invention which is reasonable and effectively overcomes the above drawbacks is provided.

### SUMMARY OF THE INVENTION

An object of the invention is to provide an air purifier, which can easily be personally portable and placed beside a user indoors or cooperating with other respiratory equipment outdoors.

An air purifier includes a housing, a filter unit and a sterilization unit. The housing has an air outlet and an air inlet under the air outlet. A filtering chamber is formed in the housing by the air outlet and the air inlet. The filtering chamber has a first chamber and a second chamber. The filter unit is disposed in the first chamber. The sterilization unit is disposed in the filtering chamber. A fan is disposed in the second chamber of the housing and over the sterilization unit. The fan is used for sucking in air from outside the housing, passing the air through the filter unit and the sterilization unit, and emitting the air. This can accomplish an object of air purification.

### BRIEF DESCRIPTION OF THE DRAWINGS

FIG. 1 is an exploded view of the invention;

FIG. 2 is an assembled view of the invention;

FIG. 3 is another assembled view of the invention;

FIG. 4 is a cross-sectional view of the invention; and

FIG. 5 is a schematic view of the invention in use.

### DETAILED DESCRIPTION OF THE INVENTION

To further disclose the features and technical contents of the invention, please refer to the following description and the drawings. However, the drawings are used for reference and description only, and do not intend to limit the invention.

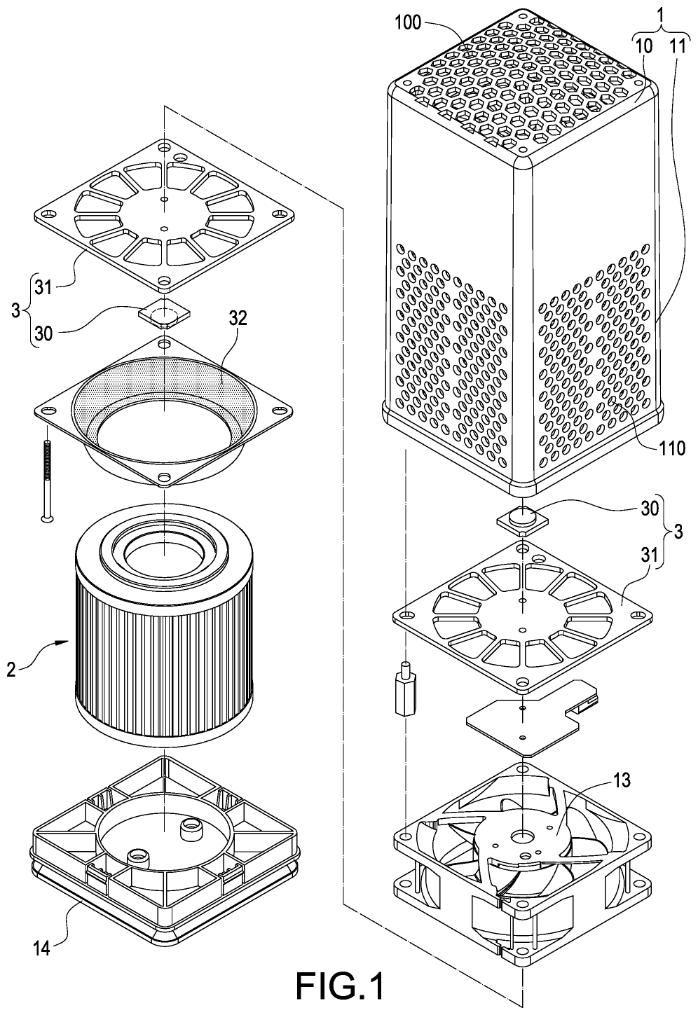

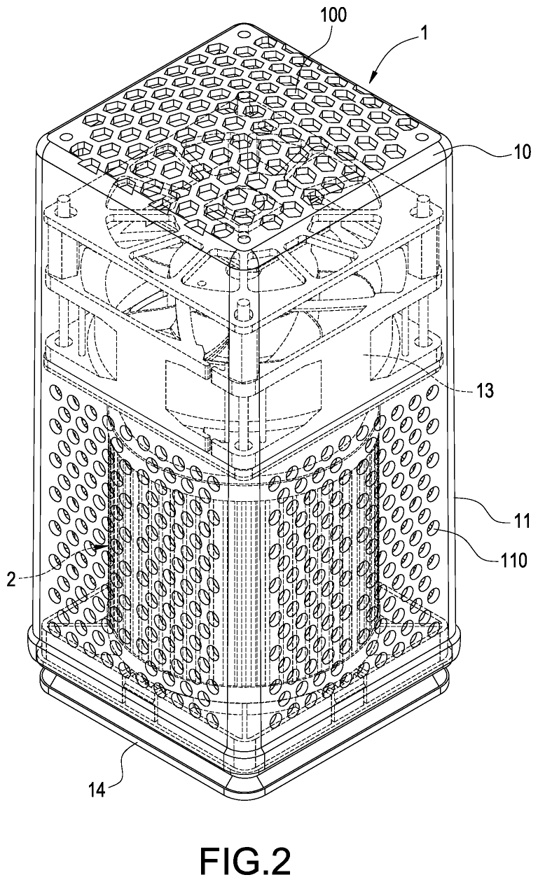

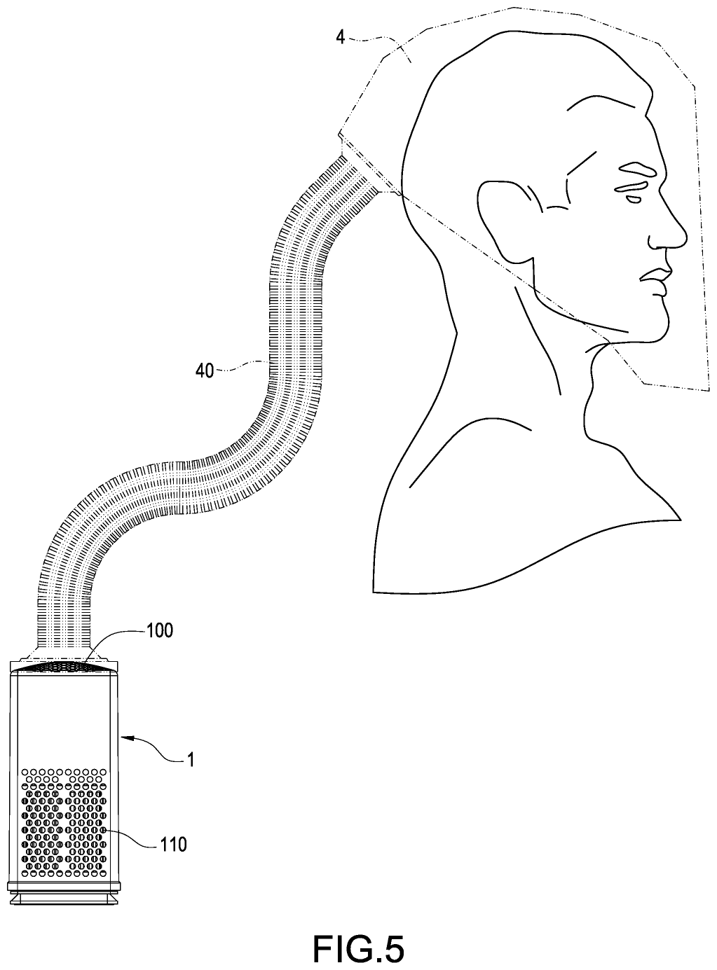

Please refer to FIGS. 1, 2 and 3, which are an exploded view, an assembled, and another assembled view of the invention. The invention provides an air purifier, which can be placed indoors or cooperate with other respiratory equipment outdoors as shown in FIG. 5. The air purifier includes a housing 1, a filter unit 2 and at least one sterilization unit 3.

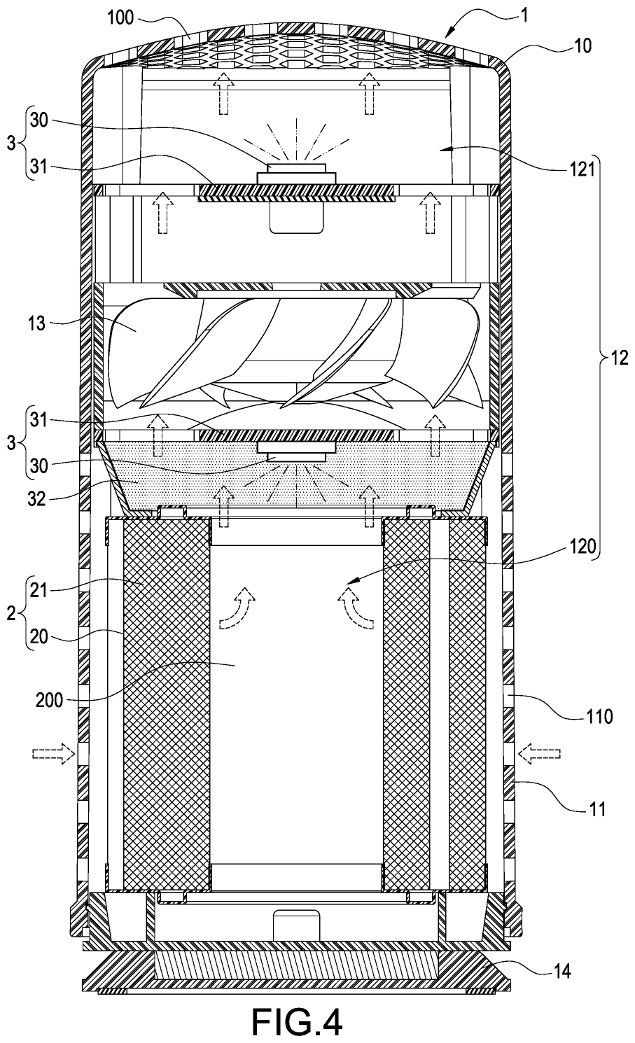

The housing 1 has an air outlet 10 and an air inlet 11 under the air outlet 10. The air outlet 10 is provided with outlet holes 100 at or near the top of the housing 1. The air inlet 11 is provided with inlet holes 110. The inlet holes 110 may surround the air inlet 11. A filtering chamber 12 is formed in the housing 1 by the air outlet 10 and the air inlet 11. As shown in FIG. 4, the filtering chamber 12 includes a first chamber 120 communicating with the inlet holes 110 and a second chamber 121 communicating with the outlet holes 100. In detail, the first chamber 120 may be located in the air inlet 11 and the second chamber 121 may be located in the air outlet 10.

A fan 13 is disposed in the housing 1. The fan 13 is located in the second chamber 121 to make the air outside the housing enter the first chamber 120 through the inlet holes 110, pass through the second chamber 121 and flow out of the housing 1 at the outlet holes 100.

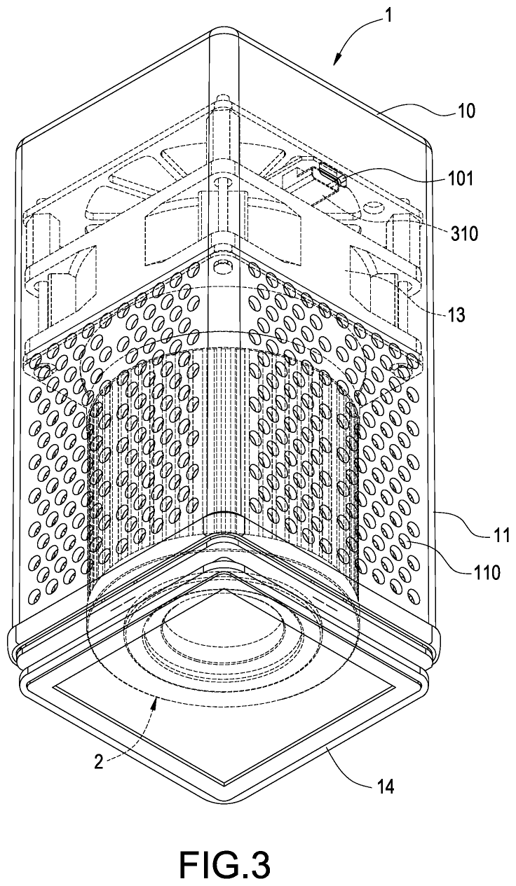

As shown in FIGS. 1-3, in this embodiment of the invention, the bottom of the housing 1 may be an opening for receiving the filter unit 2 and the sterilization unit 3. The bottom of the air inlet 11 is provided with a cover 14 for sealing the opening as shown in FIG. 3. The cover 14 is assembled at the bottom of the housing 1 and is detachable.

Please refer to FIGS. 1-4. The filter unit 2 is disposed in the first chamber 120 and includes a filter cylinder 20 and a filter structure 21 disposed in the filter cylinder 20. The filter structure 21 may be close to the inlet holes 110. The filter cylinder 20 has a hollow core 200 around which the filter structure 21 is arranged. The sterilization unit 3 is disposed in the filtering chamber 12 and under the fan 13. The sterilization unit 3 includes an ultraviolet (UV) light source 30 and a control circuit board 31. The UV light source 30 may be UV LEDs and is located under the fan 13 and between the first chamber 120 and the second chamber 121 or over the fan 13 and in the second chamber 121. In this embodiment of the invention, the sterilization unit 3 is two in number and the two sterilization units 3 are separately located over and under the fan 13 with or without contact. As shown in FIG. 4, the sterilization unit 3 over the fan 13 is superposed over the fan 13 with an interval.

Accordingly, as shown in FIG. 3, when the fan 13 is operating, the fan can drive airflow. Air outside the housing 1 enters the first chamber 120 through the inlet holes 110. The entering air passes through the filter unit 21 for filtration and is further sucked into the second chamber 121 for sterilization by either one or both of the sterilization units 3. The UV light source 30 of the sterilization unit 3 under the fan 13 shines downwards and the UV light source 30 of the sterilization unit 3 over the fan 13 shines upwards. Further, an inner wall of the UV light source 30 is coated with a photocatalyst layer 32 to enhance the effect of sterilization. Finally, clean air which has been filtered and sterilized is emitted through the outlet holes 100 to accomplish air purification.

Please refer to FIG. 3. As for the power required by the fan 13 and the sterilization unit 3, the control circuit board 31 of the sterilization unit 3 is electrically connected to a power supply connector 310. The power supply connector 310 corresponds to a power supply hole 101 of the housing 1 or the air outlet 10. Electricity can be supplied to the fan 13 by electrically connecting the control circuit board 31 and the fan 13.

Accordingly, by the abovementioned structure, an air purifier can be obtained.

Accordingly, the air purifier of the invention can be placed indoors to purify indoor air. As shown in FIG. 5, the invention may also cooperate with other respiratory equipment outdoors. The respiratory equipment includes a mask 4 and a tube 40. The tube 40 is connected to the outlet holes 100 of the invention, so that the purified air can be sent into the mask 4 for respiration. In this manner, the invention can be portably carried outdoors to supply clean air to users.

In sum, the invention is a rare product, which can accomplish the expected object to solve the conventional drawbacks. As the invention possesses novelty and is not obvious, a patent for the invention is requested.

It will be appreciated by persons skilled in the art that the above embodiments have been described by way of example only and not in any limitative sense, and that various alterations and modifications are possible without departure from the scope of the invention as defined by the appended claims.

What is claimed is:

1. An air purifier comprising:
a housing, having an air outlet and an air inlet under the air outlet, the air outlet being provided with outlet holes, the air inlet being provided with inlet holes, a filtering chamber being formed in the housing by the air outlet and the air inlet, and the filtering chamber comprising a first chamber communicating with the inlet holes and a second chamber communicating with the outlet holes;
a filter unit, disposed in the first chamber, and comprising a filter cylinder and a filter structure disposed in the filter cylinder; and
at least one sterilization unit, disposed in the filtering chamber, and comprising an ultraviolet (UV) light source and a control circuit board;
wherein a fan is disposed in the second chamber of the housing and over the sterilization unit, the fan is used to make air outside the housing enter through the inlet holes, pass through the filter unit and the sterilization unit and flow out at the outlet holes.

2. The air purifier of claim 1, wherein a bottom of the housing is an opening, and a cover is provided for sealing the opening.

3. The air purifier of claim 2, wherein the outlet holes are located at or near a top of the air outlet, and the inlet holes surround the air inlet.

4. The air purifier of claim 3, wherein the filter structure is close to the inlet holes.

5. The air purifier of claim 4, wherein the filter cylinder has a hollow core around which the filter structure is arranged.

6. The air purifier of claim 1, wherein the first chamber is located in the air inlet, and the second chamber is located in the air outlet.

7. The air purifier of claim 1, wherein the sterilization unit is located under the fan and between the first chamber and the second chamber, or over the fan and in the second chamber.

8. The air purifier of claim 1, wherein the sterilization unit is two in number, and the two sterilization units are separately located over and under the fan.

9. The air purifier of claim 8, wherein the sterilization unit under the fan shines downward, and the sterilization unit over the fan shines upward.

10. The air purifier of claim 1, wherein the control circuit board of the sterilization unit is electrically connected with a power supply connector, and the power supply connector corresponds to a power supply hole of the housing.
